(19) United States
(12) Patent Application Publication
(10) Pub. No.: US 2025/0263651 A1
(43) Pub. Date: Aug. 21, 2025

(54) MULTI-STAGE CULTURE METHODS FOR PRODUCING BIOMASS FROM FILAMNETOUS FUNGI

(71) Applicant: Terramino, Inc., Berkeley, CA (US)

(72) Inventors: Mamie Inoue, Berkeley, CA (US); Joshua Nixon, Berkeley, CA (US)

(21) Appl. No.: 18/857,147

(22) PCT Filed: Apr. 14, 2023
(86) PCT No.: PCT/US2023/065792

§ 371 Date: Oct. 15, 2024

Related U.S. Application Data
(60) Provisional application No. 63/331,651, filed on Apr. 15, 2022.

(51) Int. Cl. C12N 1/14

(52) U.S. Cl. CPC C12N 1/14

(57) ABSTRACT

Methods of growing biomass from filamentous fungi using two or more culture steps are described. The biomass produced by the methods described has a dispersed hyphal morphology and/or increased protein content. The filamentous fungi used may be of the Aspergillus genus, such as Aspergillus oryzae.

### CROSS-REFERENCE TO RELATED APPLICATIONS

This application is a § 371 national phase filing of International Patent Application No. PCT/US2023/065792 filed Apr. 14, 2023, entitled “MULTI-STAGE CULTURE METHODS FOR PRODUCING BIOMASS FROM FILAMNETOUS FUNGI”, which claims priority to U.S. Provisional Patent Application No. 63/331,651, entitled MULTI-STAGE CULTURE METHODS FOR PRODUCING BIOMASS FROM FILAMENTOUS FUNGI, filed Apr. 15, 2022, the entirety of which is hereby incorporated by reference.

### TECHNICAL FIELD

The present disclosure relates to multi-stage culture methods for producing biomass from filamentous fungi. The multi-stage culture methods described herein may produce biomass with a dispersed hyphal morphology and/or high protein content.

### BACKGROUND

Mycoproteins from edible filamentous fungi such as Aspergillus oryzae are a fiber-rich alternative protein source with a meat-like texture. This makes filamentous fungi including mycoproteins especially well-suited for use in meat alternative food products.

When growing edible filamentous fungi for use food products, is desirable to provide growing conditions that yield filamentous fungi biomass having dispersed hyphal morphology. When a dispersed hyphal morphology is achieved, food products created from the filamentous fungi biomass have better texture and improved protein content. While previous studies have reported on some conditions affecting the creation of a dispersed morphology, a need continues to exist for improved methods of growing filamentous fungi biomass having a dispersed hyphal morphology. It would also be advantageous if such methods produced biomass having high protein content.

### SUMMARY

This Summary is provided to introduce a selection of concepts in a simplified form that are further described below in the Detailed Description. This Summary, and the foregoing Background, is not intended to identify key aspects or essential aspects of the claimed subject matter. Moreover, this Summary is not intended for use as an aid in determining the scope of the claimed subject matter.

These and other aspects of the technology described herein will be apparent after consideration of the Detailed Description and FIGURES herein. It is to be understood, however, that the scope of the claimed subject matter shall be determined by the claims as issued and not by whether given subject matter addresses any or all issues noted in the Background or includes any features or aspects recited in the Summary.

In some embodiments, a method for producing biomass from filamentous fungi is described, the method generally including at least a first culture stage, a second culture stage, and a third culture stage. The first culture stage may include the steps of inoculating spores of a filamentous fungi, such as (but not limited to), Aspergillus oryzae, in a growth medium to form a culture broth, and incubating the culture broth for a first period of time. The incubation conditions of the first stage may include a pH in the range of from about 2.8 to about 3.7, a temperature in the range of from about 30 to about 35° C., and a time period in the range of from about 8 to about 16 hours. The second culture stage may include the steps of transferring the culture broth obtained from the first culture stage to a volume of fresh growth medium, and incubating the culture broth for a second period of time. The incubation conditions of the second stage may include a pH in the range of from about 4.8 to about 5.7, a temperature in the range of from about 30 to about 35° C., and a time period in the range of about 24 hours. The third culture stage may include the steps of transferring the culture broth from the second culture stage to a volume of fresh growth medium, and incubating the culture broth for a third period of time. The incubation conditions of the third stage may include a pH in the range of from about 4.8 to about 5.7, a temperature in the range of from about 30 to about 35° C., and a time period in the range of about 24 hours. The biomass produced by the methods described herein may have a dispersed hyphal morphology and/or an increased protein content.

### BRIEF DESCRIPTION OF THE DRAWINGS

Non-limiting and non-exhaustive embodiments of the disclosed technology, including the preferred embodiment, are described with reference to the following FIGURES, wherein like reference numerals refer to like parts throughout the various views unless otherwise specified.

FIG. 1 is a flow chart illustrating a multi-culture method for producing biomass from filamentous fungi according to various embodiments described herein.

### DETAILED DESCRIPTION

Embodiments are described more fully below with reference to the accompanying FIGURES, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense.

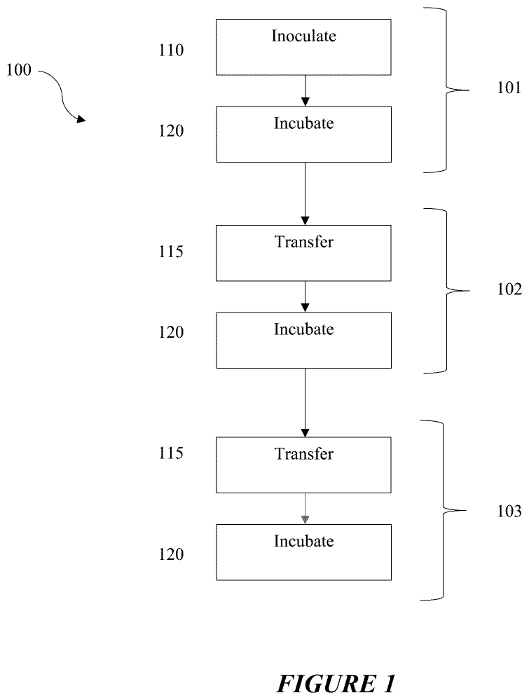

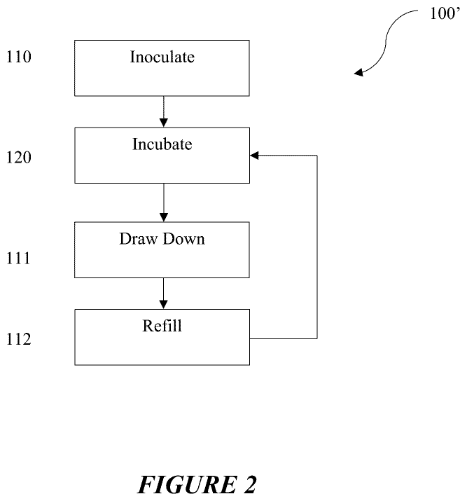

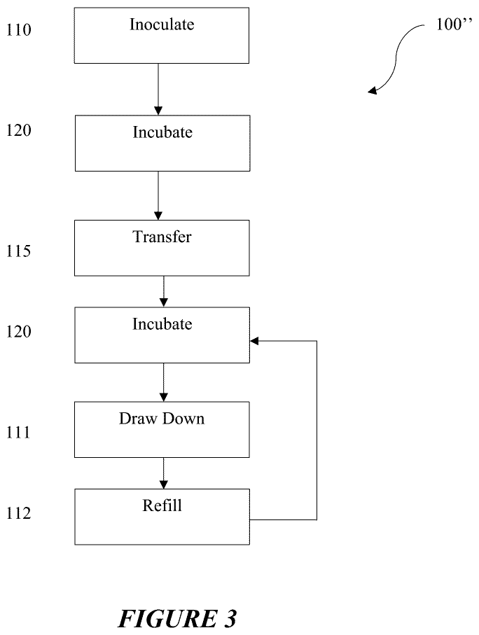

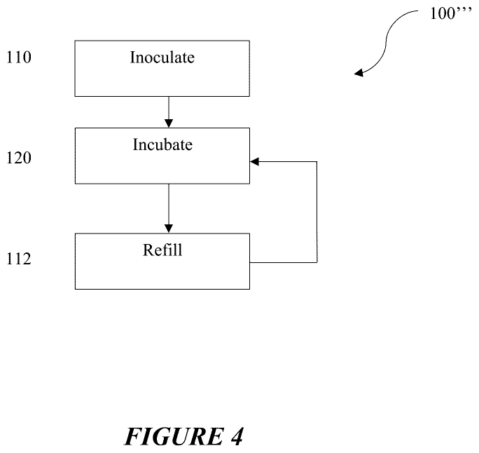

With reference to FIG. 1, a method 100 of producing a biomass from filamentous fungi may generally include a first culture stage 110, a second culture stage 120, and a third culture stage 130. The multi-culture process of the method 100 has been found to beneficially produce a biomass having dispersed hyphal morphology. The method 100 also may provide a biomass having increased protein content. In some embodiments, the biomass obtained after the third culture stage has an increase in protein content of from 50 to 60% as compared to the protein content of the biomass produced after the second culture stage.

In step 110, the first culture stage generally includes inoculating spores of a filamentous fungi in a growth medium to form a culture broth, and incubating the culture broth for a first period of time. The inoculation portion of step 110 is generally carried out using any known inoculation procedures, equipment and conditions. Generally speaking, the inoculation step will include adding the spores to a growth medium to thereby form a culture broth. The specific concentration of spores inoculated in the growth medium is not limited, though in some embodiments, a concentration in the range of from about 10⁷-10⁸ spores/mL growth medium is used.

The specific type of filamentous fungi spore used in the inoculation potion of step 110 is generally not limited. Any filamentous fungi spore suitable for use in producing a biomass can be used. In some embodiments, the filamentous fungi spore is selected from those filamentous fungi spores that produce an edible biomass such that the biomass produced by the methods described herein can be incorporated into consumable products. In one non-limiting example, the filamentous fungi spore is selected from the Aspergillus genus. In some embodiments, the filamentous fungi spore is Aspergillus oryzae.

Once the filamentous fungi spore is inoculated, step 110 further includes incubating the culture broth to begin the process of producing the biomass. In some embodiments, the incubation conditions of the first culture stage are selected to promote growth of biomass having a dispersed hyphal morphology, though the dispersed hyphal morphology is generally achieved by virtue of the complete multi-culture method described herein, as opposed to the result of just a single culture stage. In some embodiments the specific incubation conditions selected for the first culture stage include a combination of a pH in the range of from about 2.8 to about 3.7, a temperature in the range of from about 30 to about 35° C., and an incubation time period in the range of from about 8 hours to about 16 hours. The incubation is carried out under aerobic conditions. Any means for establishing the aerobic conditions can be used, such as through the use of shaken flask with foam plug.

In step 120, the culture broth resulting from step 110 is first transferred to fresh growth medium in order to carry out a second culture stage. In some embodiments, the volume of fresh growth medium to which the first stage culture broth is transferred is 9-times the volume of the culture broth (i.e., 10% v/v inoculation). The fresh growth medium may be the same growth medium as used in the first culture stage, or a different growth medium may be used. Any means of transferring the first stage culture broth to the fresh growth medium can be used.

In a further part of step 120, the second stage incubation is carried out to further grow the biomass. In some embodiments, the incubation conditions of the second culture stage are selected to promote growth of biomass having a dispersed hyphal morphology, though the dispersed hyphal morphology is generally achieved by virtue of the complete multi-culture method described herein, as opposed to the result of just a single culture stage. In some embodiments the specific incubation conditions selected for the second culture stage include a combination of a pH in the range of from about 4.8 to about 5.7, a temperature in the range of from about 30 to about 35° C., and an incubation time period in the range of about 24 hours. The incubation of the second stage is carried out under aerobic conditions. Any means for establishing the aerobic conditions can be used, such as through the use of shaken flask with foam plug.

In step 130, the culture broth resulting from step 120 is first transferred to fresh growth medium in order to carry out a third culture stage. In some embodiments, the volume of fresh growth medium to which the second stage culture broth is transferred is 9-times the volume of the culture broth (i.e., 10% v/v inoculation). The fresh growth medium may be the same growth medium as used in the first and/or second culture stages, or a different growth medium may be used. Any means of transferring the second stage culture broth to the fresh growth medium can be used.

In a further part of step 130, the third stage incubation is carried out to further grow the biomass. In some embodiments, the incubation conditions of the third culture stage are selected to promote growth of biomass having a dispersed hyphal morphology, though the dispersed hyphal morphology is generally achieved by virtue of the complete multi-culture method described herein, as opposed to the result of just a single culture stage. In some embodiments the specific incubation conditions selected for the third culture stage include a combination of a pH in the range of from about 4.8 to about 5.7, a temperature in the range of from about 30 to about 35° C., and an incubation time period in the range of about 24 hours. The incubation of the third stage is carried out under aerobic conditions. Any means for establishing the aerobic conditions can be used, such as through the use of shaken flask with foam plug.

The biomass resulting from completion of the third stage of the method 100 possesses a dispersed hyphal morphology. The term “dispersed hyphal morphology” is used to describe mycelium grown in a manner that is not clustered into amalgamations but instead is evenly distributed and not entangled with itself. This even distribution allows for better access to nutrients and faster growth. This morphology affects the texture of the final output of the process and makes it better suited for use as certain foodstuffs in terms of being able to better mimic the texture of the foodstuff. Growth speed of the biomass may also be improved due to the dispersed hyphal morphology.

As also alluded to previously, the protein content of the biomass produced at the completion of the third culture stage is improved using the methods described herein. In some embodiments, the protein content is increased 50 to 60% from the biomass at the completion of the second stage to the biomass at the completion of the third stage. This again can make the biomass produced by the method described herein well suited for use in various foodstuffs.

Each of steps 110, 120 and 130 include the use of a growth medium. As discussed previously, the growth medium used in each step may be the same, or different growth mediums may be used for one or more of these steps. In some embodiments, the growth medium is the same for each step. The growth medium is generally not limited, though in some embodiments, the growth medium is preferably Czapek-Dox growth medium in which the carbon source is sucrose and the nitrogen source is nitrate. That being said, other growth mediums using other carbon and/or nitrogen sources can be used. In one non-limiting example, a growth medium is used wherein the carbon source is a starch and the nitrogen source is ammonium.

While not illustrated in FIG. 1, it should be appreciated that method 100 may include any additional steps needed for processing the grown biomass. In some embodiments, method 100 further includes, upon completion of step 130, a step of harvesting the biomass from the culture broth. Any suitable manner of harvesting the biomass can be used, though care should be taken to not destroy or otherwise compromise the structure of the biomass formed so as to ensure the dispersed hyphal morphology is not disturbed. In some embodiments, a filtration process is used to harvest the biomass.

### EXAMPLES

Spores (10⁷-10⁸/mL) of a strain of Aspergillus oryzae were inoculated in 380 mL growth medium comprised of glucose, nitrate, and other minerals, in addition to a small volume of a dispersant, in a 2.8-L Fernbach flask with a foam plug. The pH of the culture broth was adjusted in the range of 3.3 to 3.7 with 1 M phosphoric acid. The culture broth was incubated for 14 hours in an orbital shaker incubator at 180 rpm and 32° C. The pH was not controlled during the incubation. The final pH following incubation was in the range of 5.0 to 5.4.

The 1^{st} stage culture broth was transferred to 3420 mL (9× volume) of a fresh growth medium comprised of glucose, nitrate, and other minerals and incubated in a 5-L stirred tank bioreactor aerobically at 32° C. The pH was controlled to remain at 5.5.

The 2^{nd} stage culture broth was harvested after 12-15 hours, and the 380 ml of the 2^{nd} stage culture broth was transferred to fresh growth medium and incubated in a 5-L stirred tank bioreactor aerobically at 32° C. The pH was controlled to remain at 5.5. The 3^{rd} stage culture broth was harvested after 12-18 hours.

The biomass protein was in the range of 43 wt % to 49 wt % in the following the 2^{nd} stage and was in the range of 44 wt % to 52 wt % following the 3^{rd} stage. The increase of the protein content from 2^{nd} to 3^{rd} stage was 2 to 20%.

The above procedure was carried out four times.

Table 1 provides a summary of the experimental conditions and results of the above-described experimantion.

TABLE 1

| | 1st culture | | | 2nd | | | 3rd | 2nd to |
|---|---|---|---|---|---|---|---|---|
| 1st | pH (pre- | 2nd | | culture | 3rd | | culture | 3rd |
| culture | incubation | culture | 2nd culture | biomass | culture | 3rd culture | biomass | protein |
| time | to post- | time | pH | protein | time | pH | protein | increase |
| period | incubation) | period | (controlled) | (wt %) | period | (controlled) | (wt %) | (%) |
| 14 h | 3.7 -> 5.4 | 14 h | 5.5 | 45.8% | 12 h | 5.5 | 49.6% | 8.3% |
| 14 h | 3.7 -> 5.4 | 15 h | 5.5 | 43.1% | 16 h | 5.5 | 44.0% | 2.1% |
| 14 h | 3.5 -> 5.0 | 15 h | 5.5 | 43.0% | 18 h | 5.5 | 51.7% | 20.2% |
| 14 h | 3.7 -> 5.3 | 12 h | 5.5 | 48.7% | 12 h | 5.5 | 50.3% | 3.3% |

From the foregoing, it will be appreciated that specific embodiments of the invention have been described herein for purposes of illustration, but that various modifications may be made without deviating from the scope of the invention. Accordingly, the invention is not limited except as by the appended claims.

Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention resides in the claims hereinafter appended.

Unless otherwise indicated, all number or expressions, such as those expressing dimensions, physical characteristics, etc., used in the specification (other than the claims) are understood as modified in all instances by the term “approximately”. At the very least, and not as an attempt to limit the application of the doctrine of equivalents to the claims, each numerical parameter recited in the specification or claims which is modified by the term “approximately” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all sub-ranges or individual values that are between and/or inclusive of the minimum value of 1 and the maximum value of 10; that is, all sub-ranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

1. A method for producing biomass from filamentous fungi, the method comprising:
a first culture stage comprising inoculating spores of a filamentous fungi in a growth medium to form a culture broth and incubating the culture broth for a first period of time;
a second culture stage comprising transferring the culture broth obtained from the first culture stage to a volume of fresh growth medium and incubating the culture broth for a second period of time; and
a third culture stage comprising transferring the culture broth from the second culture stage to a volume of fresh growth medium and incubating the culture broth for a third period of time.

2. The method of claim 1, wherein the culture broth of the first culture stage to incubation has a pH in the range of from about 2.8 to about 3.7 prior to incubating the culture broth of the first culture stage, and the culture broth of the first culture stage has a pH in the range of from about 5.0 to 5.5 after incubating the culture broth of the first culture stage.

3. The method of claim 1, wherein the first culture stage is carried out at a temperature in the range of from about 30 to about 35° C.

4. The method of claim 1, wherein the first time period is about 10 to about 18 hours.

5. The method of claim 1, wherein the first culture stage is carried out aerobically.

6. The method of any preceding claim 1, wherein the culture broth of the second culture stage has a pH in the range of from about 4.0 to about 5.7 after incubating the culture broth of the second culture stage.

7. The method of claim 1, wherein the second culture stage is carried out at a temperature in the range of from about 30 to about 35° C.

8. The method of claim 1, wherein the second time period is about 12 to about 24 hours.

9. The method of claim 1, wherein the second culture stage is carried out aerobically.

10. The method of claim 1, wherein the culture broth of the third culture stage has a pH in the range of from about 4.0 to about 5.7 after incubating the culture broth of the third culture stage.

11. The method of claim 1, wherein the third culture stage is carried out at a temperature in the range of from about 30 to about 35° C.

12. The method of any claim 1, wherein the third time period is about 12 to about 24 hours.

13. The method of claim 1, wherein the third culture stage is carried out aerobically.

14. The method of claim 1, wherein the spores are spores of the genus Aspergillus.

15. The method of claim 1, wherein the spores are spores of Aspergillus oryzae.

16. The method of claim 1, wherein the growth medium used in the first, second, and third culture stages includes a carbon source comprising sugar and a nitrogen source comprising an inorganic nitrate.

17. The method of claim 1, wherein the growth medium used in the first, second, and third culture stages comprises Czapek-Dox growth medium.

18. The method of claim 1, further comprising:
harvesting a biomass from the culture broth obtained from the third culture stage.

19. The method of claim 18, wherein harvesting the biomass from the culture broth obtained from the third stage comprises a filtration step.
